(19) United States
(12) Patent Application Publication
(10) Pub. No.: US 2026/0002913 A1
(43) Pub. Date: Jan. 1, 2026

(54) METHOD AND SYSTEM FOR ANALYZING HYDROCARBON-CONTAINING SAMPLES

(71) Applicant: TOTALENERGIES ONETECH, Courbevoie (FR)

(72) Inventors: Leticia MEDINA LIGIERO, Pau (FR); Béatrice MARTIN, Pau (FR)

(21) Appl. No.: 18/992,954

(22) PCT Filed: Jul. 12, 2022
(86) PCT No.: PCT/IB2022/000408

§ 371 Date: Jan. 9, 2025

(51) Int. Cl. G01N 30/06; G01N 1/40; G01N 30/16; G01N 30/88; G01N 33/28

(52) U.S. Cl. CPC G01N 30/06; G01N 1/4055; G01N 1/4077; G01N 30/16; G01N 30/88; G01N 33/2835; G01N 2001/4088; G01N 2030/062; G01N 2030/065; G01N 2030/8854

(57) ABSTRACT

The invention relates to a method and a system for analyzing one or more compounds in a hydrocarbon-containing sample. The method comprises steps of: a) injecting the sample and a first solvent into a chamber so that the one or more compounds in the sample form a precipitate in the chamber; b) passing the precipitate from the chamber to a filter device, wherein the precipitate is captured in the filter device; c) passing a second solvent through the filter device while bypassing the chamber, so as to dissolve the one or more compounds in the precipitate captured in the filter device; and d) detecting the one or more compounds downstream of the filter device. The system comprises a chamber, a sample feeding line, a first solvent feeding line, a filter device, a transfer line, a second solvent feeding line, and a detector.

### TECHNICAL FIELD

The present invention relates to a method and a system for analyzing one or more compounds in a hydrocarbon-containing sample.

### TECHNICAL BACKGROUND

Petroleum or crude oil is a complex mixture of hydrocarbons and other chemicals, whose composition widely varies depending on how and where the crude oil was formed. Among several compounds included in the crude oil, asphaltenes are known as the fraction of crude oil insoluble in alkanes (such as n-heptane or n-pentane) but soluble in aromatic solvents (such as toluene or benzene). Asphaltenes are destabilized due to changes in crude oil composition, pressure, and/or temperature conditions, and tend to precipitate during the production and/or refinery process of crude oil. Precipitation of asphaltenes has been a known problem in the oil industry as it leads to physical blockage of the line, alteration of wettability, decreased viscosity of crude oil, formation of emulsion, and fouling in equipment, for example, causing a significant loss in production efficiency.

It is thus important to analyze characteristics of asphaltenes and other compounds included in crude oil, such as stability and solubility, in the design and monitoring of different processes in the oil industry.

Document U.S. Pat. No. 10,449,502 relates to a method of analyzing chemical compositions of hydrocarbons, comprising precipitating asphaltenes from a hydrocarbon sample with a precipitant solvent in a chemically inert stationary phase, and passing eluates of precipitated asphaltenes through a size exclusion chromatography (SEC) stationary phase. This method relying on a HPLC column filled with PTFE (Polytetrafluoroethylene) suffers from a problem of adsorption of asphaltenes on PTFE beads, which is often irreversible and leads to incomplete analysis.

The problems associated with known in-column methods are discussed in another document U.S. Pat. No. 9,671,384. This document relates to a method of determining asphaltene stability in a hydrocarbon-containing sample, comprising precipitating an amount of asphaltenes from a hydrocarbon-containing liquid sample, capturing precipitated asphaltenes in a low volume filter having a porous filter element. The low volume filter is placed in-line with a chromatography and asphaltene concentration in eluted fractions from the low volume filter is monitored using a liquid chromatography detector. This document teaches that the method employing the low volume filter improves repeatability and reproducibility of analysis as compared to the known in-column methods. However, this document gives no detail about how precipitation of asphaltenes occurs, and analysis results provided by the proposed method are still insufficient. Furthermore, the inventors found that the proposed method still suffers from residual adsorption of asphaltenes on the filter, possibly due to insufficient mixing of crude samples with precipitants.

Therefore, there is a need for an improved method for analyzing precipitable compounds such as asphaltenes in crude oil, which enables repeatable, reproducible and accurate analysis in a more efficient and cost-effective manner.

### SUMMARY OF THE INVENTION

It is a first object of the invention to provide a method of analyzing one or more compounds in a hydrocarbon-containing sample, comprising steps of:
a) injecting the sample and a first solvent into a chamber so that the one or more compounds in the sample form a precipitate in the chamber;
b) passing the precipitate from the chamber to a filter device, wherein the precipitate is captured in the filter device;
c) passing a second solvent through the filter device while bypassing the chamber, so as to dissolve the one or more compounds in the precipitate captured in the filter device; and
d) detecting the one or more compounds downstream of the filter device.

According to some embodiments, the step a) comprises filling the chamber with the first solvent, and then injecting the sample into the chamber filled with the first solvent.

According to some embodiments, the method further comprises waiting for a predetermined period from 5 to 500 seconds, preferably from 10 to 200 seconds, more preferably from 20 to 60 seconds after injection of the sample, before implementing step b).

According to some embodiments, the second solvent comprises two or more different compounds, and the composition of the second solvent varies over time while passing through the filter device.

It is a second object of the invention to provide a system for analyzing one or more compounds in a hydrocarbon-containing sample, comprising:
a chamber,
a sample feeding line fluidically connected to the chamber, for feeding the sample into the chamber,
a first solvent feeding line fluidically connected to the chamber, for feeding a first solvent into the chamber,
a filter device downstream of the chamber,
a transfer line for passing fluid from the chamber to the filter device,
a second solvent feeding line fluidically connected to the filter device, for feeding a second solvent to the filter device while bypassing the chamber, and
a detector downstream of the filter device.

According to some embodiments, the chamber comprises at least one inlet, preferably two or more inlets.

According to some embodiments, the sample feeding line and the first solvent feeding line are fluidically connected to different inlets of the chamber.

According to some embodiments, the inner diameter of the at least one inlet of the chamber is from 0.05 to 1 mm, preferably from 0.1 to 0.4 mm, more preferably from 0.15 to 0.2 mm.

According to some embodiments, an inner volume of the chamber is from 0.1 to 10 mL, preferably from 0.2 to 3 mL, more preferably from 0.5 to 1.5 mL.

According to some embodiments, the chamber has a surface-to-volume ratio from 0.45 to 0.7, preferably from 0.5 to 0.65, more preferably from 0.55 to 0.6.

According to some embodiments, the chamber comprises an outlet fluidically connected to the filter device, an inner diameter of the outlet being from 0.2 to 3 mm, preferably from 0.5 to 2 mm, more preferably from 0.75 to 1.5 mm.

According to some embodiments, the system further comprises a connecting element fluidically connecting the second solvent feeding line and the transfer line to a common line, wherein the common line is fluidically connected to an inlet of the filter device.

According to some embodiments, the system further comprises a first pump fluidically connected to the first solvent feeding line and configured to make the first solvent flow through the first solvent feeding line.

According to some embodiments, the system further comprises a second pump fluidically connected to the second solvent feeding line and configured to make the second solvent flow through the second solvent feeding line.

According to some embodiments for the method or the system as described above, the one or more compounds in the sample comprise or are asphaltenes.

Therefore, the invention provides a method and a system that can provide an analysis of precipitable compounds such as asphaltenes in a hydrocarbon-containing fluid with excellent repeatability and accuracy. Further, the method and system according to the invention are cost-effective and efficient with reduced time and less consumption of sample and solvents.

This is achieved by the presence of a chamber in the system in which a precipitate of one or more compounds in a hydrocarbon-containing sample is formed. More particularly, the injected sample comes into contact with a first solvent in the chamber, and the precipitate is formed inside the chamber. Thus the precipitate of the compound(s) required for analysis may be obtained with reduced consumption of sample and solvents.

Further, a second solvent is passed through the filter device while bypassing the chamber for dissolving the compound(s) in the precipitate captured in the filter device. Namely, the second solvent enters the filter device without passing through the chamber. This minimizes the dead volume, which is defined as an extra volume experienced by solutes while passing through a system, leading to better peak resolutions.

In contrast to the in-column method as described in U.S. Pat. No. 10,449,502 which employs a filled column, the chamber for the method and the system according to the invention does not suffer from significant adsorption of compound(s). As inner surfaces of the chamber may be free of adsorption or adhesion of compound(s) in the hydrocarbon-containing sample, it is not necessary to frequently clean and/or replace the chamber.

The precipitate of the compound(s) is effectively captured in the filter device, and the captured precipitate is effectively dissolved for analysis by the second solvent entering the filter device without passing through the chamber. Namely, the precipitate is completely dissolved and carried from the filter device for analysis, and would not remain on the filter device to cause residual adsorption on the filter device as is the case in U.S. Pat. No. 9,671,384. That may lead to more accurate and reproducible analysis.

### BRIEF DESCRIPTION OF THE DRAWINGS

FIG. 1 shows a flowchart of the method according to the invention.

FIG. 2 illustrates in a schematic manner the system according to the invention.

FIG. 3A is a top perspective view of an embodiment of an assembly forming a chamber for the method and the system according to the invention.

FIG. 3B is a top plan view of the embodiment of the assembly as shown in FIG. 3A.

FIG. 3C is a cross-sectional view along the line E-E in FIG. 3B of the embodiment of the assembly.

FIG. 3D is a perspective view cut along the line E-E in FIG. 3B of the embodiment of the assembly, showing only a half part of the assembly.

FIG. 3E is an enlarged view of a part of the cross sectional view of FIG. 3C.

FIG. 3F is an enlarged view of a part of the cross sectional view of FIG. 3C.

FIG. 4A is a graph illustrating the temporal variation of the second solvent composition in the first example.

FIG. 4B is a graph illustrating the temporal variation of the second solvent composition in the second example.

FIG. 5 is a graph illustrating the detection results in the first example.

FIG. 6 is a graph illustrating the detection results in the second example.

FIG. 7 is a graph illustrating the detection results in a comparative example.

### DESCRIPTION OF EMBODIMENTS

The invention will now be described in more detail without limitation in the following description.

### Method of Analyzing

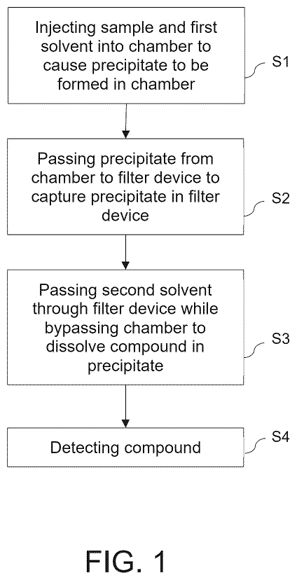

With reference to FIG. 1, the method of analyzing one or more compounds in a hydrocarbon-containing sample according to the invention is described.

The method comprises a step of injecting the hydrocarbon-containing sample into a chamber (S1). For the purpose of the invention, the term “hydrocarbon-containing sample” means any crude or processed sample containing hydrocarbon materials, including but not limited to, crude oils, petroleum products such as heavy oils, petroleum cokes, tars and asphalts, oil sands, oil shales, shale oils, and coal liquefaction products.

The sample may be a crude or raw sample, or a prepared or treated sample. For example, the sample may be diluted in any appropriate solvent or solution before injection. In some embodiments, the sample is prepared before injection by being diluted in the same solvent as the second solvent which is to be described later.

The volume of sample injected into the chamber may range for example from 0.1 μL to 1 mL, preferably from 0.2 μL to 500 μL, or from 0.3 μL to 200 μL, or from 0.5 μL to 100 μL, or from 0.7 μL to 50 μL. In some embodiments, from 1 to 15 μL, preferably from 2 to 10 μL, more preferably from 4 to 8 μL of the sample is injected into the chamber.

In step S1, a first solvent is also injected into the chamber. In some embodiments, the first solvent is injected simultaneously with the sample, via the same inlet or a separate inlet different from the inlet for injecting the sample. Alternatively, the first solvent may be injected temporally separately from the sample, for example via the same inlet or via a separate inlet from the inlet for injecting the sample. Preferably, the first solvent is injected first to fill the chamber, followed by injecting the sample (whether via the same or different inlet) into the chamber filled with the first solvent. In any case, injection of the sample and the first solvent is performed so that the sample comes into contact with the first solvent only after entering the chamber.

The sample injected into the chamber comes into contact with the first solvent in the chamber, which brings one or more compounds in the sample to form a precipitate. In some embodiments, the one or more compounds in the sample comprise or are asphaltenes, and a precipitate of asphaltenes is formed in the chamber. However, the method according to the invention may be used for other precipitable compounds, such as analyzing deposits of other compounds included in the hydrocarbon-containing sample, such as saturates, aromatics or resins.

For the purpose of the invention, the term “asphaltenes” means the most polar components of crude oil, which are organic heterocyclic macromolecules comprising carbon, hydrogen, oxygen, nitrogen, and sulfur. Asphaltenes are insoluble in normal alkanes (such as normal pentane and heptane, for example) but soluble in aromatics (such as benzene and toluene, for example).

The first solvent may comprise any appropriate solvent selected depending on the one or more compounds in the sample intended to precipitate for analysis. For example, the first solvent may comprise one or more compounds, selected from straight-chain or branched-chain alkanes ranging in carbon number from 3 (propane) to 10 (n-decane), such as iso-octane, n-pentane, n-hexane or n-heptane, petroleum ether, ethyl acetate, alcohols and combinations thereof. The one or more compounds of the first solvent are miscible with each other.

In some embodiments, the first solvent is injected so that the sample-to-first solvent volume ratio within the chamber is low. For example, this ratio may range from 1:1000 to 1:10, preferably from 1:500 to 1:20, more preferably from 1:200 to 1:50, more preferably from 1:150 to 1:75, such as, by way of example only, approximately 1:100.

In some embodiments, the method further comprises a step of waiting for a predetermined period after injection of the sample (or after injection of the sample and the first solvent in the case of simultaneously injecting both). In an embodiment, the flow of the sample and/or the first solvent into the chamber may be temporarily stopped while waiting for the predetermined period. The flow of the sample and/or the first solvent may be resumed after the predetermined period of waiting. In an alternative embodiment, the method may dispense with the step of waiting for the predetermined period and/or temporary suspension of the flow of the sample and/or the first solvent. The predetermined period may be appropriately set so that the precipitate of the one or more compounds in the sample is formed in the chamber. For example, the predetermined period is from 5 to 500 seconds, preferably from 10 to 200 seconds, more preferably from 20 to 60 seconds.

The method then comprises a step of passing the precipitate, which is formed in the chamber, from the chamber to a filter device (S2). The precipitate is carried from the chamber to the filter device, and captured in the filter device.

Subsequently, the method comprises a step of passing a second solvent through the filter device while bypassing the chamber (S3). Namely, the second solvent is injected to pass through the filter device without passing through the chamber.

The second solvent passing through the filter device dissolve the one or more compounds in the precipitate captured in the filter device.

The second solvent may comprise any appropriate solvent selected depending on the one or more compounds in the precipitate intended to dissolve for analysis. For example, the second solvent may comprise one or more compounds selected from aromatic solvents such as benzene, toluene, xylene, and methyl naphthalene, dichloromethane (DCM), cyclohexane, tetrahydrofuran (THF), chloroform, trichloroethylene, tetrachloroethylene, carbon tetrachloride, carbon disulfide, pentane, isopropanol, heptane, methanol and combinations thereof. The one or more compounds of the second solvent are miscible with each other.

In some embodiments, the second solvent may comprise two or more different compounds, and the composition of the second solvent may vary over time while passing through the filter device. The composition of the second solvent may be varied over time in a continuous manner, in an incremental or stepwise manner, or in an intermittent manner. Particularly, the second solvent may be passed through the filter device while varying the volume ratio between the different compounds in the second solvent over time. More specifically, the second solvent may comprise two or more compounds A and B (and so on), and the second solvent may be passed through the filter device while varying the volume ratio of A relative to B over time. Alternatively, the second solvent may comprise three or more compounds A, B, and C (and so on), and the compounds A, B, and C may be injected in two or more successive steps. Each of the compounds A, B, and C may be a single solvent or a mixture of solvents. Injection of the second solvent may also comprise injection of the same one or more compounds more than once, for example a sequence of injecting compounds A, B, then A or a sequence of A, B, C, then A. Such variation of the second solvent makes it possible to gradually dissolve different compounds in the precipitate and thus detect them separately.

For example, the second solvent may comprise heptane and toluene, and may be passed through the filter device while varying the volume ratio of heptane relative to toluene over time, for example from 100 to 0 in a stepwise manner, which may optionally be followed by injection of isopropanol while linearly increasing the volume ratio of isopropanol relative to toluene over time, for example from 0 to 100. Alternatively, the second solvent may comprise heptane and DCM, and may be passed through the filter device while varying the volume ratio of heptane relative to DCM over time, for example from 100 to 0 in a stepwise manner, which may optionally be followed by injection of methanol while linearly increasing the volume ratio of methanol relative to DCM over time, for example from 0 to 50.

In some embodiments, the second solvent comprises at least the same compound as the first solvent. For example, the first solvent comprises heptane and the second solvent comprises at least heptane, or the first solvent comprises pentane and the second solvent comprises at least pentane. In some embodiments, the second solvent comprising at least the same compound as the first solvent is passed through the filter device so that injection of the second solvent starts with the same compound as the first solvent, then followed by injection of other one or more different compounds of the second solvent, while varying the volume ratio over time as described above.

In some embodiments, the step of passing the second solvent through the filter device to dissolve the precipitate takes from 1 to 300 min, preferably from 10 to 200 min, or from 20 to 120 min, or from 30 to 100 min, more preferably from 40 to 80 min. The duration for dissolution may be appropriately set according to the conditions of analysis.

Flow rates for injecting the sample and the first solvent may be defined appropriately so that the residence time permits the compound(s) in the sample to form a precipitate in the chamber and to be carried to the filter device. Similarly, flow rates for injecting the second solvent may be defined appropriately so that the precipitate of the one or more compounds in the sample captured in the filter device is dissolved according to the need for subsequent analysis.

In some embodiments, the sample, the first solvent, and/or the second solvent are injected respectively with a flow rate from 0.01 to 2 mL/min, preferably from 0.1 to 1 mL/min, such as for example of approximately 0.5 mL/min. The flow rates for injecting the sample, the first solvent, and the second solvent may be the same or different.

The method then comprises a step of detecting the one or more compounds downstream of the filter device (S4). Namely, the compound(s) dissolved in the second solvent is/are detected downstream of the filter device.

Detection may be performed by any appropriate method using any appropriate device, depending on the compound(s) intended to be analyzed. Examples of devices for such detection include, but not limited to, a charged aerosol detector (CAD), a mass spectrometer, an evaporative light scattering detector (ELSD), and any detector available for the use in conjunction with chromatography.

In some embodiments, detection provides a chromatogram which makes it possible to determine the concentrations of one or more compounds in the dissolved precipitate and thus the concentration of these compounds in the sample. Particularly, the second solvent comprising two or more different compounds may be passed through the filter device with gradients as mentioned above, and detection may provide a chromatogram on which different peaks or patterns correspond to different compounds in the dissolved precipitate.

In some embodiments, the step of detecting comprises determining the contents and/or characteristics of the one or more compounds in the sample, such as one or more parameters of solubility, stability, density and viscosity, concentrations, ratios between two or more different compounds, and combinations thereof.

In some embodiments, the method optionally comprises a step of injecting one or more further additives. Examples of such additives include, but not limited to, asphaltene inhibitors, detergents, antioxidants, stabilizers, metal deactivators, viscosity/friction modifiers, demulsifying agents, dispersants, anti-foam agents, and combinations thereof. As such, the method according to the invention may also be used for testing such one or more further additives. Particularly, the method according to the invention enables more efficient and effective analysis of additives compared to the bottle test which is commonly known for assessing oil emulsion stability or for evaluating the effectiveness of asphaltene inhibitors. Indeed, the bottle test generally requires about 100 mL of samples and takes about 8 h for screening. Further, the bottle test is not reliable as the results are evaluated by visual check which is highly operator dependent. The bottle test also requires a cleaning step which usually consumes time and solvents. The method according to the invention may be advantageously used in combination with a HPLC system to enable more efficient analysis. The analysis steps may be automated and controlled by the system, and take a shorter amount of time, such as less than 2 h, for example approximately 1 h with less volume of sample.

The one or more further additives may be added at any point in the method according to the invention. For example, the additive(s) may be mixed with the sample and/or the first solvent before injecting into the chamber, injected into the chamber together with the sample and/or the first solvent (via the same or different inlet), injected into the chamber separately from the sample and/or the first solvent (via the same or different inlet), injected into the filter device together with the second solvent (via the same or different inlet), and/or injected into the filter device separately from the second solvent (via the same or different inlet).

In some embodiments, the sample is injected routinely or periodically while the first solvent passes through the chamber continuously. Namely, all or part of the steps of the method according to the invention, from the step S1 to the step S4, may be repeated several times. For example, the steps S1 to S2 may be repeated more than one time, followed by a single sequence of the steps S3 to S4.

With the method according to the invention, the precipitate is effectively formed in the chamber, carried to the filter device by the first solvent passing through the chamber, and captured in the filter device. The captured precipitate is effectively dissolved by the second solvent passing through the filter device without passing through the chamber. Thus, the method according to the invention enables accurate and reproducible analysis, and effectively prevents undesirable adsorption on the inner surface of the chamber and on the filter device, and fouling or clogging in the chamber.

### System

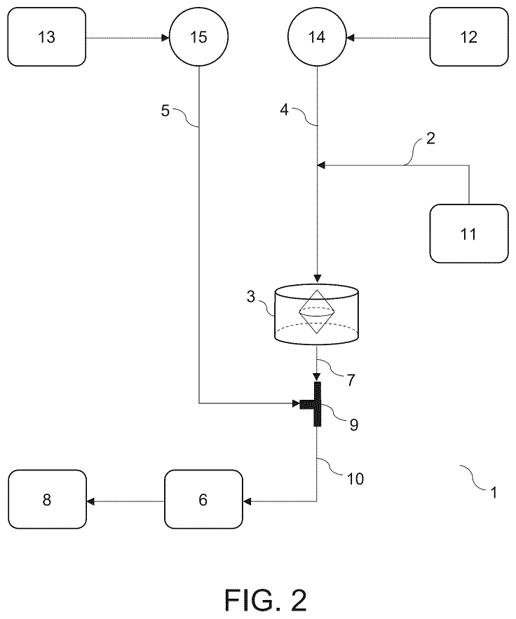

Next, the system for analyzing one or more compounds in a hydrocarbon-containing sample according to the invention is described with reference to FIG. 2. The system of the invention may be used to implement the method of the invention. The method of the invention may be implemented in the system of the invention.

The definition for the hydrocarbon-containing sample, the one or more compounds in the sample, the first solvent and the second solvent is to be referred to the above description for the method according to the invention. Indeed, all the definitions/statements given above for the features of the method according to the invention are also applied to the system according to the invention. Similarly, all the definitions/statements given below for the features of the system according to the invention are also applied to the method according to the invention as described above.

The system 1 comprises a chamber 3. The chamber is to be discussed in more detail below.

The system 1 comprises a sample feeding line 2 for feeding the hydrocarbon containing sample into the chamber 3. The sample feeding line 2 is fluidically connected to the chamber 3. Preferably, the sample feeding line 2 is connected to one or more sources of the hydrocarbon containing sample 9.

The system 1 further comprises a first solvent feeding line 4 for feeding a first solvent into the chamber 3. The first solvent feeding line 4 is fluidically connected to the chamber 3. Preferably, the first solvent feeding line 4 may be connected to one or more sources of the first solvent 12.

The system 1 further comprises a filter device 6 downstream of the chamber 3. In some embodiments, the filter device 6 comprises one filter, or two or more filters arranged in series. The pore diameter of the filter(s) may be selected appropriately so as to capture the precipitate of the one or more compounds. For example, the pore diameter of the filter(s) may be from 0.1 to 0.6 μm, preferably from 0.2 to 0.5 μm. Particularly, the filter device 6 may comprise one filter of 0.2 μm or 0.5 μm, or two filters of 0.2 μm arranged in series. The filter device 6 may be made of any appropriate material compatible with the solvents used. Examples of the material of the filter device 6 include, but not limited to, stainless steel, gold, titanium, silver, gold coated stainless steel, titanium coated stainless steel or silver coated stainless steel, carbon composite, nickel-containing alloys, polyaryletherketones, polytetrafluoroethylene, and the combinations thereof. The filter device 6 may be any commercially available filter, including but not limited to, EXP® Pre-Column Filter Cartridges and OPTI-SOLV® In-Line Filter Replacement Frits, both available from Optimize Technologies, for example.

The system 1 further comprises a transfer line 7 for passing fluid from the chamber 3 to the filter device 6.

The system 1 further comprises a second solvent feeding line 5 for feeding a second solvent to the filter device 6 while bypassing the chamber 3. Namely, the second solvent feeding line 5 is configured to feed the second solvent to the filter device 6 without passing the second solvent through the chamber 3. The second solvent feeding line 5 is fluidically connected to the filter device 6.

The system 1 further comprises a detector 8 downstream of the filter device 6. As defined above, the detector 8 may be any detector available for the use in conjunction with chromatography.

In some embodiments, the system 1 further comprises a connecting element 9 which fluidically connects the second solvent feeding line 5 and the transfer line 7 to a common line 10. The common line 10 is fluidically connected to an inlet of the filter device 6. The connecting element 9 is downstream of the chamber 3. Specifically, the precipitate is carried from the chamber 3 via the connecting element 9 to the filter device 6, and the second solvent is passed through the filter device 6 via the connecting element 9. The second solvent feeding line 5 may be configured to feed the second solvent to the filter device 6 via the connecting element 9. Preferably, the connecting element 9 comprises an inlet through which the second solvent is injected. The connecting element 9 may comprise an outlet, and the common line 10 may fluidically connect the outlet of the connecting element 9 and the inlet of the filter device 6.

In some embodiments, the sample feeding line 2, the first solvent feeding line 4, and/or the second solvent feeding line 5 may possibly comprise any intermediate elements such as a pump, an injecting device, a selection valve, and combinations thereof. For example, as illustrated in FIG. 2, the system 1 may comprise a first pump 14 which is fluidically connected to the first solvent feeding line 4 and used for feeding the first solvent from the source 12 to the chamber 3. Similarly, the system 1 may comprise a second pump 15 which is fluidically connected to the second solvent feeding line 5 and used for feeding the second solvent from the source 13 to the filter device 6. However, the number and position of pumps are not limited and the system 1 may comprise any other intermediate elements, for example a dedicated pump for injecting the sample, and/or an injector for injecting both the sample and the first solvent into the chamber 3. For example, the discharge pressure of the pump used for feeding the first solvent and/or the second solvent may be of from 20 to 100 bar, preferably from 30 to 50 bar, such as for example approximately 40 bar. Possibly the system may also comprise an injection apparatus or system which controls injection of the sample, the first solvent and/or the second solvent in an automated manner according to the predetermined setting, such as an autosampler.

In some embodiments, the chamber 3 comprises at least one inlet. The chamber may comprise only one inlet through which both the sample and the first solvent are injected into the chamber. Namely, the sample feeding line 2 and the first solvent feeding line 4 may be fluidically connected to the same single inlet of the chamber 3. In other embodiments, the chamber 3 may comprise two or more, for example three or four inlets. In the case of the chamber 3 having two or more inlets, the sample and the first solvent may be injected through the same inlet, or through separate inlets respectively. Namely, the sample feeding line 2 and the first solvent feeding line 4 may be fluidically connected to the same inlet, or to separate inlets from each other. In the case of the chamber having two or more inlets, one or more inlets of the chamber 3 separate from the inlet for injecting the sample and/or the first solvent may be used for injecting one or more further additives.

### Chamber

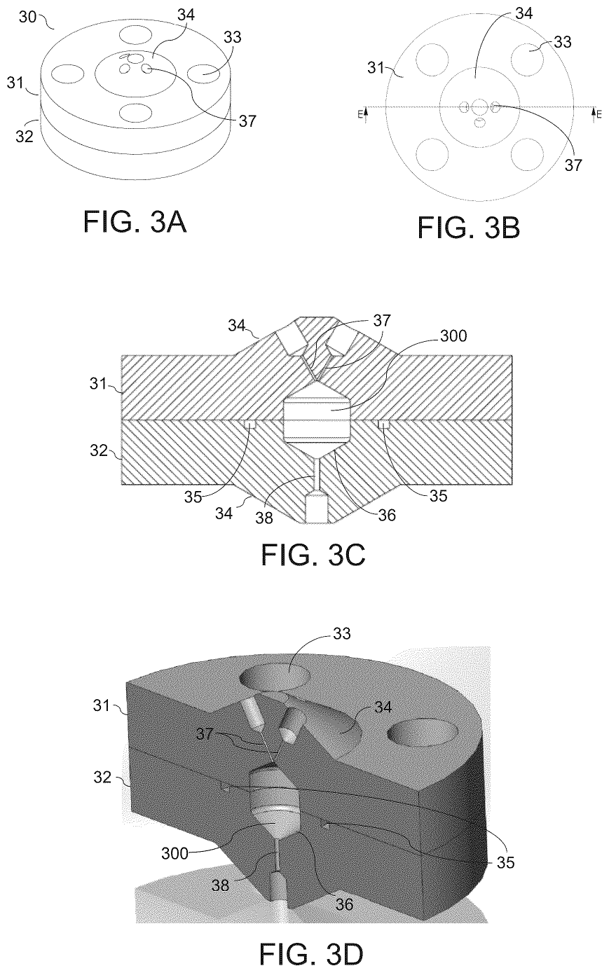

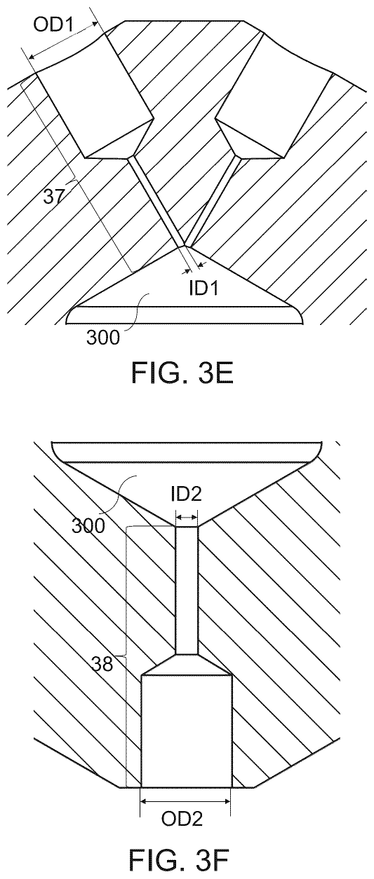

The chamber for the method and the system according to the invention is now discussed in detail with reference to FIGS. 3A to 3F.

FIG. 3A shows a top perspective view of an embodiment of an assembly 30 which forms the chamber 300 for the method and the system according to the invention. FIG. 3B shows a top plan view of the embodiment of the assembly 30. FIG. 3C shows a cross-sectional view along the line E-E in FIG. 3B of the embodiment of the assembly 30. FIG. 3D shows a perspective view cut along the line E-E in FIG. 3B of the embodiment of the assembly 30, showing only a half part of the assembly 30. FIGS. 3E and 3F respectively show an enlarged view of a part of the cross sectional view of FIG. 3C.

In the example embodiment illustrated in FIGS. 3A to 3D, a first element 31 and a second element 32 are assembled together to form a piece of assembly 30, in which the chamber 300 is formed as an inner space of the piece of assembly 30, between the first element 31 and the second element 32 (the chamber 300 is not shown in FIGS. 3A and 3B). However, one single piece of element may also be provided in which the chamber 300 is formed as an inner hollow space or cavity.

When the chamber is formed by the first element 31 and the second element 32 assembled together, the first and second elements 31, 32 can be conveniently disassembled to see whether there is any adsorption on an inner surface of the chamber, or to clean the inner surface of the chamber, for example.

For example, the first and second elements 31, 32 may be assembled with the use of one or more bolts, nuts, screws or any other suitable fixing elements, clamps, glue or adhesive bonds, and combinations thereof. The first and second elements 31, 32 may comprise one or more screw holes or threaded bores for receiving such fixing element(s). For example, FIG. 3A shows four holes 33 for receiving fixing elements on the top surface of the first element 31. However, the number and position of such holes for receiving fixing element(s) are not limited.

A joint, packing, gasket, washer or any other sealing 35 may optionally be provided at the boundary or interface between the first and second elements 31, 32, as seen in FIGS. 3C and 3D.

In the example embodiment illustrated in FIGS. 3A to 3D, the first element 31 and the second element 32 are respectively configured as a cylinder having a convex portion 34 around the center on an outer surface. However, the shape or geometry of the first and second elements 31, 32 are not limited but the first and second elements 31, 32 may have any other suitable shape such as a disk, a saucer-like shape or a polygonal prism.

The outer surfaces of the first and second elements 31, 32 are surfaces which face the outside when assembled together. Namely, when assembled together, the outer surfaces of the first and second elements 31, 32 form an outer contour of the entire assembly 30. The convex portions 34 around the center on the outer surfaces of the first and second elements 31, 32 may be configured as a hemisphere, a polygon or a geodesic dome, for example.

Inner surfaces of the first and second elements 31, 32 are surfaces which face with each other and form the chamber 300 when assembled together. Namely, the first and second elements 31, 32 are assembled so that the respective inner surfaces face with each other. Each of the first and second elements 31, 32 has a concave portion or hollow 36 on the inner surface. The concave portions or hollows 36 on the inner surfaces of the first and second elements 31, 32 are configured such that, when the first and second elements 31, 32 are assembled together, the concave portions 36 integrally form the chamber 300 inside the assembly 30. Particularly, assembled together, the inner surface of the concave portion 36 of the first element 31 and the inner surface of the concave portion 36 of the second element 32 form a substantially seamless and integral inner surface of the chamber 300.

The first element 31 and the second element 32 may not be necessarily symmetrical, but may be designed independently as long as the chamber 300 is formed when the two elements are assembled together. Preferably, the concave portions 36 on the inner surfaces of the first and second elements 31, 32 are designed and positioned symmetrically. In the example embodiment illustrated in FIGS. 3A to 3D, the first and second elements 31, 32 are designed to have the same shape and dimension, in other words two units or copies of the same element 31, 32 are provided and assembled to form a one piece of assembly 30.

In some embodiments, the chamber 300 is designed as an empty, void space surrounded by the inner surfaces of the first and second elements 31, 32, more specifically by the inner surfaces of the concave portions 36 of the first and second elements 31, 32. With the term “empty” and “void”, it is meant that the chamber 300 does not have any obstructing feature such as blades, vanes, baffles and plates. Such empty or void chamber 300 may effectively suppress possible adsorption of compound(s) in the chamber 300, otherwise any obstructing feature inside the chamber would provide more surfaces which would favor the possible adsorption thereon.

For example, the chamber 300 may be configured as a cylinder or a prism having an n-sided polygon base, with one or both ends selected from a hemisphere, a cone, a frustum, and a pyramid. However, the chamber 300 is not limited to any specific shape.

Preferably, the chamber 300 has an inner surface area as small as possible relative to its inner volume. In some embodiments, the chamber 300 has a surface-to-volume ratio of from 0.45 to 0.7, preferably from 0.5 to 0.65, more preferably from 0.55 to 0.6. For the purpose of the invention, the surface-to-volume ratio of the chamber is defined as the amount of inner surface area per unit inner volume of the chamber. The chamber 300 having the inner surface area as small as possible compared to the inner volume can advantageously suppress possible adsorption or adhesion of compound(s) in the chamber 300.

In some embodiments, the chamber 300 has an inner volume of from 0.1 to 10 mL, preferably from 0.2 to 3 mL, more preferably 0.5 to 1.5 mL.

In some embodiments, the chamber 300 comprises at least one inlet 37 which is open to the outside on the outer surface of the first element 31 or the second element 32. In the example embodiment illustrated in FIGS. 3A to 3D, the chamber 300 comprises three inlets 37 which are open to the outside on the outer surface of the first element 31. However, the number and position of inlets 37 are not limited but the chamber 300 may have only one inlet, three or four inlets, for example.

In some embodiments, the chamber 300 comprises an outlet 38 which is open to the outside on the outer surface of the first element 31 or the second element 32. If the inlet(s) 37 is provided so as to be open to the outside on the outer surface of the first element 31, the outlet 38 is provided so as to be open to the outside on the outer surface of the second element 32, and vice versa. For example, FIGS. 3C and 3D show two inlets 37 open to the outside on the outer surface of the first element 31 and the outlet 38 open to the outside on the outer surface of the second element 32.

In some embodiments, the at least one inlet 37 of the chamber 300 has an inner diameter of from 0.05 to 1 mm, preferably from 0.1 to 0.4 mm, more preferably from 0.15 to 0.2 mm. In the case of the chamber having two or more inlets 37, the inner diameters of the inlets may be the same or different.

In some embodiments, the inlet(s) 37 of the chamber 300 may be configured as a tube or a channel extending through the first or second element 31, 32, having an outer end open to the outside on the outer surface of the first or second element 31, 32 and an inner end open to the chamber 300. Such inlet(s) 37 configured as a tube or a channel may have the same inner diameter over the entire length of the tube or channel. Namely, the inlet(s) 37 may have the same, constant inner diameter from the inner end to the outer end. In other embodiments, the inlet(s) 37 may be configured to have varying inner diameters over the entire length from the inner end to the outer end of the inlet(s) 37. For example, the outer end of the inlet(s) 37 may have a larger inner diameter than the inner diameter of the inner end of the inlet(s) 37. Such arrangement may be useful for connecting the chamber 300 to the lines, which may be made of tubing, and facilitate injection of the sample and/or the first solvent into the chamber 300. Such example embodiment is illustrated in FIG. 3E, which is an enlarged view of a part of the cross-sectional view of FIG. 3C showing the inlets 37 and a part of the chamber 300. As illustrated in FIG. 3E, the inner diameter (OD1) of the outer end of the inlet(s) 37 may be from 1 to 5 mm, preferably from 1.25 to 3 mm, more preferably from 1.5 to 2 mm. Namely, in the example embodiment with varying inner diameters along the inlet(s) 37, the inner diameter of the inlet(s) 37 may vary in a range from 0.1 to 5 mm, so that at least the inner diameter (ID1) of the inner end of the inlet(s) 37 which is open to the chamber 300 may be from 0.1 to 1 mm, preferably from 0.2 to 0.7 mm, more preferably from 0.3 to 0.45 mm. In the example embodiment illustrated in FIGS. 3C to 3E, the inner diameter (OD1) of the outer half of the inlet 37 is larger than the inner diameter (ID1) of the inner half of the inlet 37.

As seen in FIGS. 3C to 3E, the outer end of the inlet(s) 37 is open to the outside on the outer surface of the first element 31, and the inner end of the inlet(s) 37 is fluidically connected to the chamber 300. Specifically, the inlet(s) 37 is configured such that the sample and/or first solvent may be injected via the outer end of the inlet(s) 37, passing through the inlet(s) 37 to enter the chamber 300 via the inner end of the inlet(s) 37.

In some embodiments, the outlet 38 of the chamber 300 has an inner diameter of from 0.2 to 3 mm, preferably from 0.5 to 2 mm, more preferably from 0.75 to 1.5 mm.

In some embodiments, the outlet 38 of the chamber 300 may be configured as a tube or a channel extending through the first or second element 31, 32, having an outer end open to the outside on the outer surface of the first or second element 31, 32 and an inner end open to the chamber 300. Such outlet 38 configured as a tube or a channel may have the same inner diameter over the entire length of the tube or channel. Namely, the outlet 38 may have the same, constant inner diameter from the inner end to the outer end. In other embodiments, the outlet 38 may be configured to have varying inner diameters over the entire length from the inner end to the outer end of the outlet 38. For example, the outer end of the outlet 38 may have a larger inner diameter than the inner diameter of the inner end of the outlet 38. Such arrangement may facilitate connection between the chamber 300 and a downstream element, for example the filter device. Such example embodiment is illustrated in FIG. 3F, which is an enlarged view of a part of the cross-sectional view of FIG. 3C showing the outlet 38 and a part of the chamber 300. As illustrated in FIG. 3F, the inner diameter (OD2) of the outer end of the outlet 38 may be from 1 to 5 mm, preferably from 1.25 to 3 mm, more preferably from 1.5 to 2 mm. Namely, in the example embodiment with varying inner diameters along the outlet 38, the inner diameter of the outlet 38 may vary in a range from 0.2 to 5 mm, so that at least the inner diameter (ID2) of the inner end of the outlet 38 which is open to the chamber 300 may be from 0.2 to 3 mm, preferably from 0.5 to 2 mm, more preferably from 0.75 to 1.5 mm. In the example embodiment illustrated in FIGS. 3C, 3D and 3F, the inner diameter (OD2) of the outer half of the outlet 38 is larger than the inner diameter (ID2) of the inner half of the outlet 38.

Whether the inner diameter of the outlet 38 is constant or varied from the inner end to the outer end, the outer end of the outlet 38 preferably has the same inner diameter with that of a tubing which is to be connected to the outer end of the outlet 38. Such tubing may fluidically connect the outlet 38 of the chamber 300 to the filter device, or to the connecting element (if any) downstream of the chamber 300.

As seen in FIGS. 3C, 3D and 3F, the outer end of the outlet 38 is open to the outside on the outer surface of the second element 32, and the inner end of the outlet 38 is fluidically connected to the chamber 300. Specifically, the outlet 38 is configured such that the sample, the first solvent and/or the resulting precipitate may exit the chamber 300 from the inner end of the outlet 38, passing through the outlet 38 and are carried to the downstream element such as the filter device from the outer end of the outlet 38.

In some embodiments, the chamber 300 is made of inox. For example, the first and second elements 31, 32 may be made of inox to form the chamber 300 of inox. Alternatively, the first and second elements 31, 32 may be made of any other material and at least the inner surfaces or parts which form the chamber 300 when assembled have a lining or coating of inox, so that at least the chamber 300 as assembled is surrounded by the inner surface of inox. The chamber made of inox is compatible with the organic solvents.

### EXAMPLES

The following examples illustrate the invention without limiting it.

### Example 1

Measurements were performed on samples A and B of different crude oils prepared as described below.

Sample A was prepared by dissolving 0.2 g of crude oil A in 100 mL of toluene.

Sample B was prepared by dissolving 0.2 g of crude oil B in 100 mL of toluene.

1 mL of each sample was put in a HPLC vial and placed on the autosampler of the system.

The system according to the invention is of the type as illustrated in FIG. 2. Specifically, the system comprised a chamber of the type as illustrated in FIGS. 3A-3F. The system comprised a first pump for feeding a first solvent and a second pump for feeding a second solvent. The system further comprised a T-shaped connecting element with three ends, the first end fluidically connected to an outlet of the chamber via a transfer line, the second end fluidically connected to the second pump via a second solvent feeding line, and the third end fluidically connected to an inlet of a filter device.

Two EXPR Pre-Column Filter Cartridges with a 0.2 μm pore size (available from Optimize Technologies) arranged in series were used as the filter device.

The chamber was made of inox and comprised three inlets and one outlet. The inner volume of the chamber was 1 mL. For the three inlets, the inner diameter of the outer end exposed to the outside was 1.50 mm, and the inner diameter of the inner end fluidically connected to the chamber was 0.38 mm. For the outlet, the inner diameter of the outer end exposed to the outside was 1 mm.

First, heptane as the first solvent was injected into the chamber at a flow rate of 0.6 mL/min for 2 min, or until the chamber is completely full of heptane and there are no leaks in the system, using the first pump.

After, 2 μL of the sample was injected into the chamber using an injector, and the flow of the sample was stopped and the chamber was left to stand for 1 min for allowing precipitates to be formed in the chamber. The total time between injection and flow interruption depends on the volume needed to carry the sample from the injector to the chamber. After waiting for 1 min, the flow of heptane was resumed for carrying the formed precipitates from the chamber to the filter device.

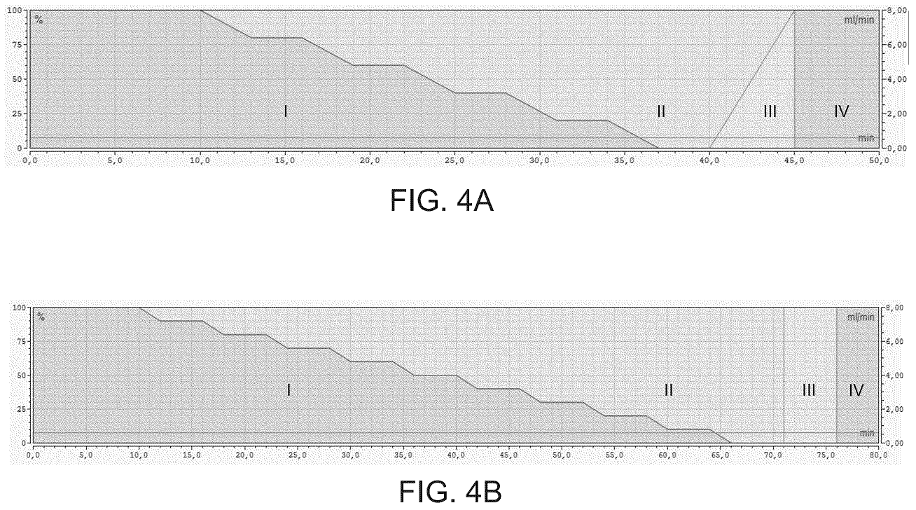

Then, the second solvent was injected into the filter device at a flow rate of 0.6 mL/min for 50 min using the second pump. During 50 min of the injection, the composition of the second solvent was varied over time as follows and as illustrated in FIG. 4A:
Injection started with 100% heptane for 10 min.
During 3 min, heptane was linearly decreased by 20%, i.e. from 100 to 80%, while toluene was linearly increased by 20%, i.e. from 0 to 20%.
Injection continued with 80% heptane and 20% toluene for 3 min.
The stepwise sequence consisting of linear decrease of heptane by 20% and linear increase of toluene by 20% during 3 min with an interval of 3 min was repeated for up to 37 min after the start, at which time the composition of the second solvent was 0% heptane and 100% toluene,
Injection continued with 100% toluene for 3 min.
During 5 min, toluene was linearly decreased from 100 to 0% while isopropanol was linearly increased from 0 to 100%.
Injection continued with 100% heptane for 5 min until the end.

In FIG. 4A, the left vertical axis indicates the percentage (%) of each compound in the second solvent flow, the right vertical axis indicates the flow rate (mL/min), and the horizontal axis indicates the time (min). Labels I, II, III, IV indicate heptane, toluene, isopropanol and heptane, respectively.

The precipitates dissolved in the second solvent was detected using a charged aerosol detector (CAD) operated at the following conditions:

For this work, CAD evaporator temperature was set at 40° C., Nitrogen (60 psi) was used for nebulization; a noise filter of 5 s and a data collection of 10 Hz were used. No inverse gradient was used.

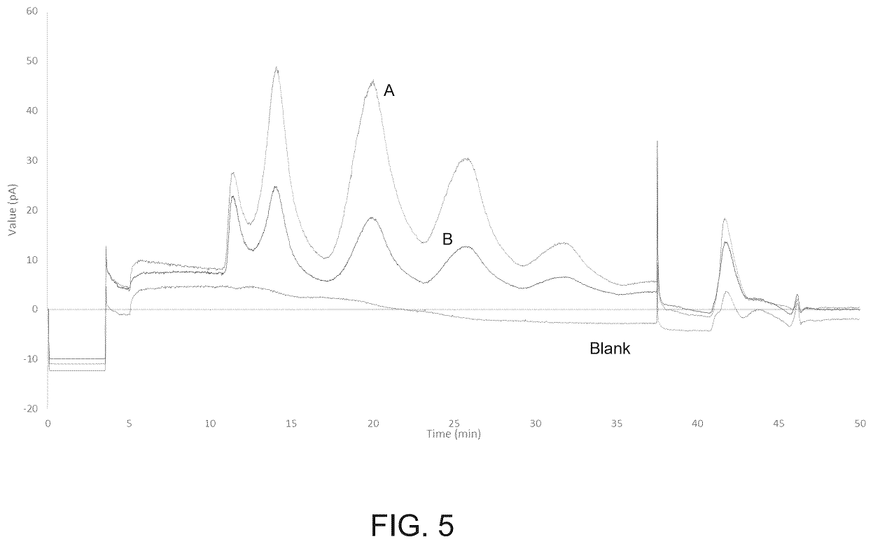

FIG. 5 shows the obtained response for the samples A (upper trace) and B (lower trace). The bottom trace indicates a blank sample for injection of the solvents only without any sample. In FIG. 5, the vertical axis indicates the detector's response value in picoampere (pA), and the horizontal axis indicates the time (min).

FIG. 5 shows five different pics that correspond to different types of asphaltenes. Peaks 1 and 2 (12 and 15 min) are asphaltenes that are soluble in a solvent composed from 100% heptane to a solution 80:20 v. % volume of heptane: toluene (first plateau). Peak 3 represents asphaltenes that are soluble in a solvent composed from 80:20 v. % heptane: toluene to a solution 60:40 v. % of heptane: toluene (second plateau). Peak 4 represents asphaltenes that are soluble in a solvent composed from 60:40 v. % heptane: toluene to a solution 40:60 v. % of heptane: toluene (third plateau). Finally, peak 5 represents asphaltenes that are soluble in a solvent composed from 40:60 v. % heptane: toluene to a solution 80:20 v. % of heptane: toluene (forth plateau).

The method using the precipitation chamber according to the present invention allows a really good separation of asphaltenes molecules in function of the solubility on the used solvent or mixture. The results can also be used for quantitative analysis with a proper quantitative method.

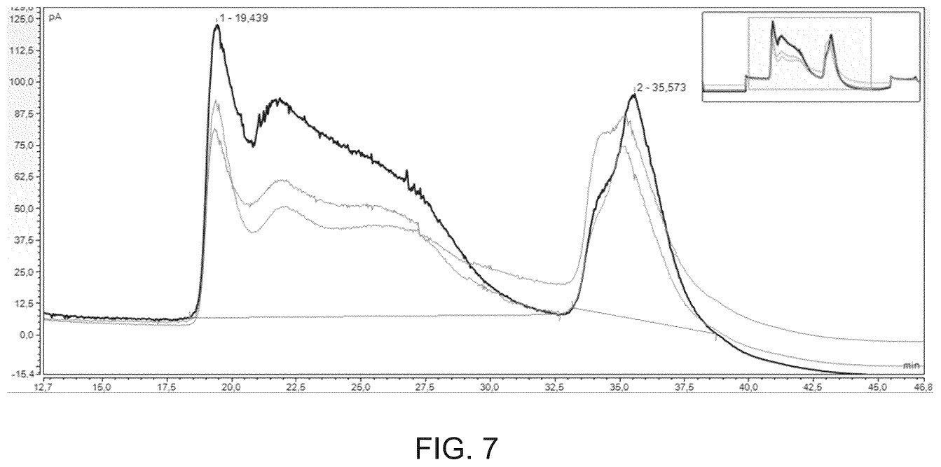

FIG. 7 shows a comparative example using the on-column precipitation method in U.S. Pat. No. 9,671,384. A gradient form 100% heptane to a 100% dicloromethane is applied from 17 min to 27 min. Then a second gradient from 100% dichloromethane to 100% methanol is applied from 30 to 40 min.

It is possible to observe that the on-column method with the applied gradient in U.S. Pat. No. 9,671,384 did not allow the separation by solvent quality as occurred in the method according to the present invention.

### Example 2

Measurements were performed on samples C, D and E of different crude oils prepared as described below.

Sample C was prepared by dissolving 0.2 g of crude oil C in 100 mL of toluene.

Sample D was prepared by dissolving 0.2 g of crude oil D in 100 mL of toluene.

Sample E was prepared by dissolving 0.2 g of crude oil E in 100 mL of toluene.

Measurements were performed with the same system and under the same conditions as that of example 1, except that the second solvent was injected for 80 min with the composition varied over time as follows and as illustrated in FIG. 4B:
Injection started with 100% heptane for 10 min.
During 2 min, heptane was linearly decreased by 10%, i.e. from 100 to 90%, while toluene was linearly increased by 10%, i.e. from 0 to 10%.
Injection continued with 90% heptane and 10% toluene for 4 min.
the stepwise sequence consisting of linear decrease of heptane by 10% and linear increase of toluene by 10% during 2 min with an interval of 4 min was repeated for up to 66 min after the start, at which time the composition of the second solvent was 0% heptane and 100% toluene.
Injection continued with 100% toluene for 5 min.
Injection continued with 100% isopropanol for 5 min.
Injection continued with 100% heptane for 4 min until the end.

In FIG. 4B, the left vertical axis indicates the percentage (%) of each compound in the second solvent flow, the right vertical axis indicates the flow rate (mL/min), and the horizontal axis indicates the time (min). Labels I, II, III, IV indicate heptane, toluene, isopropanol and heptane, respectively.

The precipitates dissolved in the second solvent was detected using a charged aerosol detector (CAD) operated at the same conditions as in example 1.

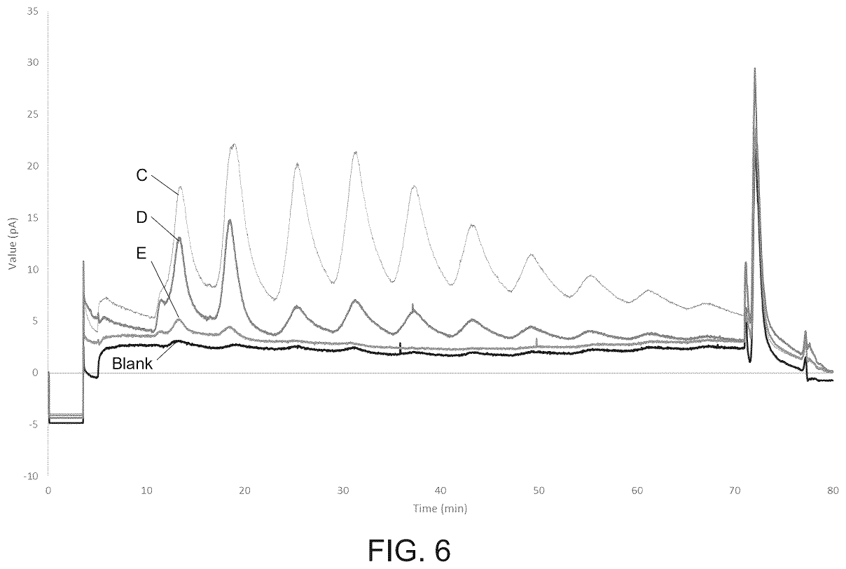

FIG. 6 shows the obtained response for the samples C (upper trace), D (middle trace) and E (lower trace). The bottom trace indicates a blank sample for injection of the solvents only without any sample. In FIG. 6, the vertical axis indicates the detector's response value in picoampere (pA), and the horizontal axis indicates the time (min).

FIG. 6 shows that for samples C and D there can be as many peaks as there are plateaus on the solvent gradient. The results are in agreement with the concept of asphaltenes: a continuum of different molecules present in the crude oil that have different solubilities in a mixture of different solvents. For samples C and D, there are as many peaks as there are plateau values on the solvent gradient, i.e. ten peaks.

Sample E shows only two peaks, they are both in the region from 100% heptane to 80% heptane, 20% toluene. This crude oil contains less asphaltenes than the other ones. Moreover, no asphaltenes were detected on the region from 80% heptane to 0% heptane.

Results on FIG. 6 shows that different crude oil samples can be compared as a function of the solubility of their asphaltenes. Quantitative results are also possible with a proper quantitative method.

1. A method of analyzing one or more compounds in a hydrocarbon-containing sample, comprising steps of:
a) injecting the sample and a first solvent into a chamber so that the one or more compounds in the sample form a precipitate in the chamber;
b) passing the precipitate from the chamber to a filter device, wherein the precipitate is captured in the filter device;
c) passing a second solvent through the filter device while bypassing the chamber, so as to dissolve the one or more compounds in the precipitate captured in the filter device; and
d) detecting the one or more compounds downstream of the filter device.

2. The method according to claim 1, wherein the step a) comprises filling the chamber with the first solvent, and then injecting the sample into the chamber filled with the first solvent.

3. The method according to claim 1, further comprising waiting for a predetermined period from 5 to 500 seconds after injection of the sample, before implementing step b).

4. The method according to claim 1, wherein the second solvent comprises two or more different compounds, and the composition of the second solvent varies over time while passing through the filter device.

5. A system for analyzing one or more compounds in a hydrocarbon-containing sample, comprising:
a chamber,
a sample feeding line fluidically connected to the chamber, for feeding the sample into the chamber,
a first solvent feeding line fluidically connected to the chamber, for feeding a first solvent into the chamber,
a filter device downstream of the chamber,
a transfer line for passing fluid from the chamber to the filter device,
a second solvent feeding line fluidically connected to the filter device, for feeding a second solvent to the filter device while bypassing the chamber, and
a detector downstream of the filter device.

6. The system according to claim 5, wherein the chamber comprises at least one inlet.

7. The system according to claim 6, wherein the sample feeding line and the first solvent feeding line are fluidically connected to different inlets of the chamber.

8. The system according to claim 6, wherein the inner diameter of the at least one inlet of the chamber is from 0.05 to 1 mm.

9. The system according to claim 5, wherein an inner volume of the chamber is from 0.1 to 10 mL.

10. The system according to claim 5, wherein the chamber has a surface-to-volume ratio from 0.45 to 0.7.

11. The system according to claim 5, wherein the chamber comprises an outlet fluidically connected to the filter device, an inner diameter of the outlet being from 0.2 to 3 mm.

12. The system according to claim 5, further comprising a connecting element fluidically connecting the second solvent feeding line and the transfer line to a common line, wherein the common line is fluidically connected to an inlet of the filter device.

13. The system according to claim 5, further comprising a first pump fluidically connected to the first solvent feeding line and configured to make the first solvent flow through the first solvent feeding line.

14. The system according to claim 5, further comprising a second pump fluidically connected to the second solvent feeding line and configured to make the second solvent flow through the second solvent feeding line.

15. The method according to claim 1, wherein the one or more compounds in the sample comprise or are asphaltenes.

16. The system according to claim 5, wherein the one or more compounds in the sample comprise or are asphaltenes.

17. The method according to claim 1, further comprising waiting for a predetermined period from 10 to 200 seconds after injection of the sample, before implementing step b).

18. The method according to claim 1, further comprising waiting for a predetermined period from 20 to 60 seconds after injection of the sample, before implementing step b).

19. The system according to claim 5, wherein the chamber comprises two or more inlets.

20. The system according to claim 5, wherein an inner volume of the chamber is from 0.5 to 1.5 mL.
